(19) United States
(12) Patent Application Publication
(10) Pub. No.: US 2010/0324551 A1
(43) Pub. Date: Dec. 23, 2010

(54) LAPAROSCOPIC SHAFT ARTICULATION BY MEANS OF A ROTATING COLLAR

(75) Inventor: Thomas J. Gerhardt, Littleton, CO (US)

Correspondence Address: TYCO Healthcare Group LP;Attn: IP Legal, 5920 Longbow Drive, Mail Stop A36, Boulder, CO, 80301-3299, US

(73) Assignee: TYCO Healthcare Group LP

(21) Appl. No.: 12/487,231

(22) Filed: Jun. 18, 2009

(51) Int. Cl. A61B 18/14

(52) U.S. Cl. 606/41

(57) ABSTRACT

A surgical instrument includes a handle and an elongated shaft extending distally from the handle. The elongated shaft includes a proximal portion coupled to the handle and a distal portion pivotally coupled to the proximal portion. An end effector is coupled to the distal portion of the elongated shaft and is articulatable relative to a longitudinal axis defined by the instrument. A cam member is rotatably mounted about the longitudinal axis and configured to impart a force to the distal portion of the elongated shaft upon rotation of the cam member to effect pivotal motion of the distal portion of the elongated shaft, and thus articulation of the end effector. An articulation actuator is supported on the handle and is operable to impart rotational motion to the cam member.

### BACKGROUND

1. Technical Field

The present disclosure relates to a surgical apparatus for laparoscopic and endoscopic procedures. In particular, the disclosure relates to a surgical apparatus having a mechanism for articulating an end effector with respect to an axis.

2. Background of Related Art

Typically) in a laparoscopic, an endoscopic, or other minimally invasive surgical procedure, a small incision or puncture is made in a patient's body. A cannula is then inserted into a body cavity through the incision, which provides a passageway for inserting various surgical devices such as scissors, dissectors, retractors, or similar instruments. To facilitate operability through the cannula, instruments adapted for laparoscopic surgery typically include a relatively narrow shaft supporting an end effector at its distal end and a handle at its proximal end. Arranging the shaft of such an instrument through the cannula allows a surgeon to manipulate the handle from outside the body to cause the end effector to carry out a surgical procedure at a remote internal surgical site. This type of laparoscopic procedure has proven beneficial over traditional open surgery due to reduced trauma, improved healing and other attendant advantages.

An articulating laparoscopic or endoscopic instrument may provide a surgeon with a range of operability suitable for a particular surgical procedure. The instrument may be configured such that the end effector may be aligned with a longitudinal axis of the instrument to facilitate insertion through a cannula, and thereafter, the end effector may be caused to articulate, pivot or move off-axis as necessary to appropriately engage tissue. When the end effector of an articulating instrument comprises a pair of jaw members for grasping tissue, a flexible control wire may be provided to open or close the jaws. The control wire may extend through an outer shaft from the handle to the jaws such that the surgeon may create a tension in the control wire to cause the jaws to move closer to one another. The closure or clamping force generated in the jaws may be directly related to the tension in the control wire applied by the surgeon.

One type of laparoscopic or endoscopic instrument is intended to generate a significant closure force between jaw members to seal small diameter blood vessels, vascular bundles or an, two layers of tissue with the application electrosurgical or RF energy. The two layers may be grasped and clamped together by the jaws of an electrosurgical forceps, and an appropriate amount of electrosurgical energy may be applied through the jaws. In this way, the two layers of tissue may be fused together. The closure forces typically generated by this type of procedure may present difficulties when using a typical control wire to open and close the jaws of an articulating instrument.

For example, a surgeon's efforts to position the jaws may be frustrated by a tendency for a control wire under tension to realign the jaws with the axis of the instrument after the jaws have been articulated off-axis. Although this tendency may be observed in any type of articulating instrument, the tendency is particularly, apparent when the closure forces and necessary tension in the control wire are relatively high, as is common in an electrosurgical sealing instrument. Significant ergonomic considerations thus manifest for an articulation actuation mechanism in an instrument such as an electrosurgical tissue sealer. These ergonomic considerations include responsiveness to a surgeon's controls and the tactile feedback provided by the instrument.

### SUMMARY

The present disclosure describes a surgical instrument including a handle for controlling the surgical instrument and an elongated shaft extending distally from the handle. The elongated shaft includes a proximal portion coupled to the handle and a distal portion pivotally coupled to the proximal portion. The proximal portion has a longitudinal axis defined therethrough. An end effector is coupled to the distal portion of the elongated shaft, and the end effector is articulatable relative to the longitudinal axis as the distal portion of the elongated shaft pivots relative to the proximal portion of the elongated shaft. A cam member is mounted such that the cam member may rotate about the longitudinal axis. The cam member is configured to impart a force to the distal portion of the elongated shaft as the cam member rotates to cause the distal portion of the elongated shaft to pivot. An articulation actuator is supported on the handle and may be operated to cause the cam member to rotate.

The articulation actuator may include a rotating collar coupled to a cam driver extending through the proximal portion of the elongated shaft. The cam driver may be configured to rotate about the longitudinal axis to cause the cam member to rotate, and the rotating collar may be mounted for rotation about the longitudinal axis. Alternatively, the rotating collar may be mounted with an oblique orientation with respect to the longitudinal axis. The rotating collar may be coupled to a drive pulley, the cam driver may be coupled to a follower pulley, and the follower pulley may be coupled to the drive pulley by a flexible belt.

The proximal portion of the elongated shaft may include a proximal joint member at a distal end thereof, and the distal portion of the elongated shaft may include a distal joint member at a proximal end thereof. The distal joint member may be pivotally coupled to the proximal joint member, and the proximal and distal joint members may exhibit a molded plastic construction.

The cam member may engage a first cam follower such that the first cam follower moves longitudinally in a distal direction in response to rotational motion of the cam member in a first direction. The first cam follower may engage the distal portion of the elongated shaft at a lateral distance from a pivot point to impart the force to distal portion of the elongated shaft. The cam member may engage a second cam follower such that the second cam follower moves longitudinally in a distal direction in response to rotational motion of the cam member in a second direction. The second cam follower may engage the distal portion of the elongated shaft at a lateral distance from the pivot point on an opposite side of the pivot point than the first cam follower to impart the force to the distal portion of the elongated shaft. The first and second cam followers may maintain engagement with both the cam member and the distal portion of the elongated shaft as the cam member rotates in the first and second directions.

The cam driver may rotate within an outer shaft tube having a lateral slot defined therein. The cam driver may be coupled to a pin projecting through the lateral slot such that the lateral slot defines the limits of travel for the collar.

The elongated shaft may include a double-joint wherein the cam member defines a central segment coupled between proximal and distal segments, wherein the proximal and distal segments pivot relative to the central segment when the central segment rotates.

The end effector may include a pair of jaw members configured to move between an open position substantially spaced from one another and a closed position wherein the jaw members are closer together. One or both of the jaw members may be coupled to a source of electrical energy.

According to another aspect of the disclosure, an electrosurgical instrument includes a handle adapted to control the instrument and an elongated shaft extending distally from the handle. The elongated shaft has a longitudinal axis defined therethrough. The end effector is pivotally coupled to a distal end of the elongated shaft such that the end effector may articulate relative to the longitudinal axis. The end effector includes a pair of jaw members configured to move between an open position and a closed position, and one or both of the jaw members is connectable to a source of electrosurgical energy. A cam member is rotatably mounted about the longitudinal axis adjacent the distal end of the elongated shaft. The cam member is configured rotate to articulate the end effector. A cam driver is rotatably mounted about the longitudinal axis and extends between the handle and the cam member. The cam driver is coupled to the cam member to impart rotational motion to the cam member. An articulation actuator is supported on the handle and is configured to impart rotational motion to the cam driver.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is a perspective view of a surgical instrument including an articulating elongated shaft in accordance with an embodiment of the present disclosure;

FIG. 2A is a partial, cross-sectional view of the elongated shaft of FIG. 1 in a first configuration aligned with a longitudinal axis;

FIG. 2B is a front view of a rotating collar in a first orientation for maintaining the elongated shaft of FIG. 2A in the first configuration;

FIG. 2C is a partial, cross-sectional view of the elongated shaft in a second configuration articulated off-axis;

FIG. 2D is a front view of the rotating collar in a second orientation for maintaining the elongated shaft in the second configuration;

FIGS. 3A through 3C are partial, cross-sectional views of the various tubular components of the elongated shaft;

FIGS. 3D through 3G are various orthogonal views of components utilized to form a joint in the elongated shaft;

FIGS. 4A and 4B are top views of joint components according to another embodiment of the present disclosure;

FIG. 4C is a partial, cross-sectional view of the joint component of FIG. 4A installed in a tubular component and supporting a rotating cam member therein;

FIG. 4D is a partial, cross-sectional view of the joint component of FIG. 4B installed in a tubular component;

FIG. 4F is a top view the cam member of FIG. 4C;

FIG. 4G is a partial, side view of the cam member;

FIG. 5A is a side view of a double-joint of an elongated shaft aligned with a longitudinal axis in accordance with another aspect of the disclosure;

FIG. 5B is a side view of the double-joint of FIG. 5A in an articulated configuration; and

FIG. 6 is a perspective view of a pulley drive mechanism having a rotating collar for driving articulation of an elongated shaft in accordance with an embodiment of the present disclosure.

### DETAILED DESCRIPTION

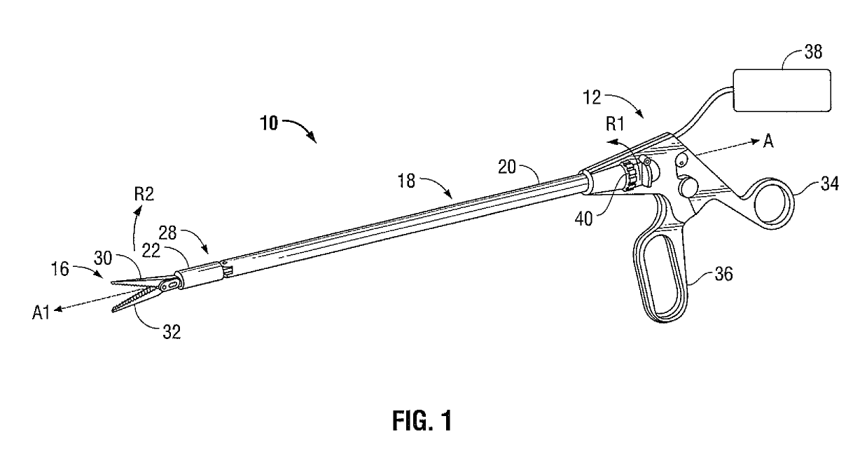

Referring initially to FIG. 1, an articulating endoscopic instrument is depicted generally as 10. The instrument 10 includes a handle 12 near a proximal end, an end effector 16 near a distal end and an elongated shaft 18 therebetween. Elongated shaft 18 includes a proximal portion 20 extending from the handle 12 and a distal portion 22 supporting the end effector 16. The proximal portion 20 defines a longitudinal axis A-A, and is sufficiently long to position the end effector 16 through a cannula (not shown). A joint 28 established between the proximal and distal portions 20, 22 of the elongated shaft 18 permits the distal portion 22 and the end effector 16 to articulate or pivot relative to the longitudinal axis A-A as described in greater detail below.

The end effector 16 includes a pair of opposing jaw members 30 and 32. The jaw members 30, 32 are operable from the handle portion 12 to move between an open configuration to receive tissue, and a closed configuration (not shown) to clamp the tissue and impart an appropriate clamping force thereto. When the end effector 16 is in the open configuration, a distal portion of each of the jaw, members 30, 32 is spaced from the distal portion of the other of the jaw members 30, 32. When the end effector 16 is in the closed configuration, the distal portions of the jaw members 30, 32 are closer together. The end effector 16 is configured for bilateral movement wherein both jaw members 30 and 32 move relative to the distal portion 22 of the elongated shaft 18 as the end effector 16 is moved between the open and closed configurations. However, unilateral motion is also contemplated wherein one of the jaw members, e.g., 32 remains stationary relative to the distal portion 22 of the elongated shaft 18 and the other of the jaw members, e.g., 30 is moveable relative to the distal portion 22.

Handle 12 is manipulatable by the surgeon from outside a body cavity to control the movement of the end effector 16 positioned inside the body at a tissue site. For example, the surgeon may separate and approximate a pivoting handle 34 relative to a stationary handle 36 to respectively open and close jaw members 30, 32. Handle 12 is also coupled to a source of electrical energy 38 such that the electrical energy may be transmitted through the elongated shaft 18 to the end effector 16. At least one of the jaw members 30, 32 is in electrical communication with the source of electrical energy 38 such that the electrical energy may be transmitted through tissue clamped between the jaw members 30, 32. The combination of mechanical clamping force applied to tissue by closing the jaw members 30, 32 and the application of electrosurgical energy through the tissue has been demonstrated to join adjacent layers of tissue captured between the jaws. A detailed discussion of the use of an electrosurgical instrument may be found in U.S. Pat. No. 7,255,697 to Dycus et al.

Handle 12 may also be manipulated to articulate the end effector 16 relative to the longitudinal axis A-A. Handle 12 supports a rotating collar 40, which is mounted about the longitudinal axis A-A. The rotating collar 40 may be rotated to the right (from the perspective of the surgeon) in the direction of arrow “R1” to articulate the end effector 16 in the direction of arrow “R2,” again to the right. Embodiments are also contemplated in which rotating collar 40 may be rotated to the left to articulate the end effector 16 to the left.

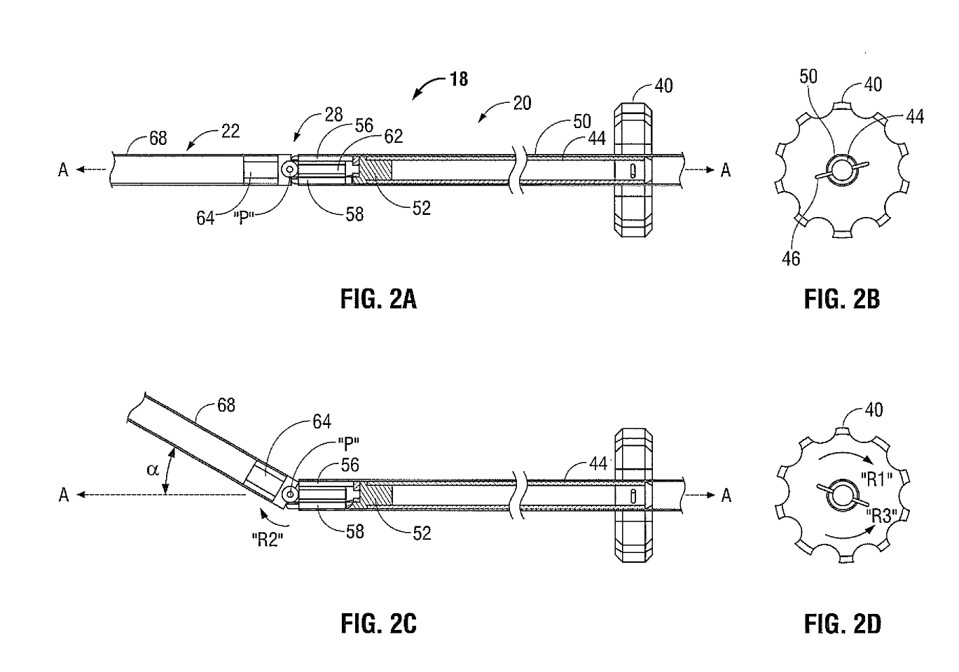

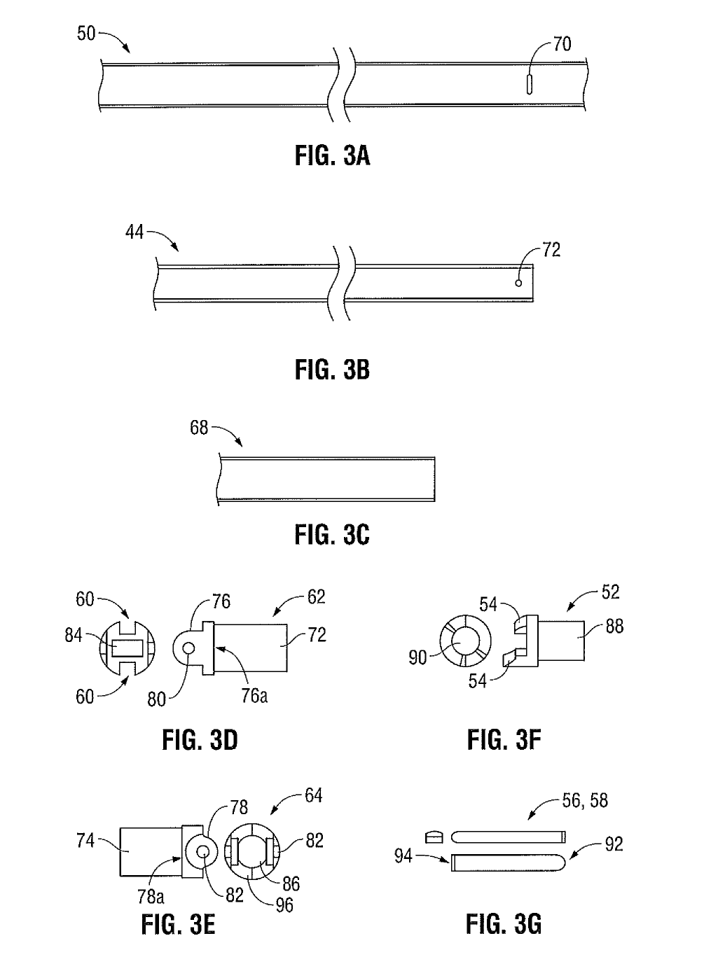

Referring now to FIGS. 2A and 2B, rotating collar 40 is rotatably mounted to the proximal portion 20 of the elongated shaft 18. The rotating collar 40 is fixedly mounted to a cam drive tube 44 with pin 46 such that rotational motion in the rotating collar 40 is transmitted to the cam drive tube 44. The can drive tube 44 rotates within an outer shaft tube 50, which extends distally to the joint 28. A cam member 52 is fixedly coupled to a distal end of the cam drive tube 44 and rotates along therewith in the outer shaft tube 50. The cam member 52 includes sloped forward cam surfaces 54 (see FIG. 3F) that engage a pair of earn followers 56, 58 such that the cam followers 56, 58 slide longitudinally in response to rotational motion in the cam member 52. Thus, a surgeon may impart longitudinal motion in the cam followers 56, 58 by rotating the collar 40.

The cam followers 56 and 58 slide within guide slots 60 (see FIG. 3D) defined in a proximal joint member 62. The proximal joint member 62 is fixedly mounted to a distal end of outer shaft tube 50. Similarly, a distal joint member 64 is fixedly mounted to a proximal end of an outer shaft tube 68. The outer shaft tube 68 supports the end effector 16 (FIG. 1). The proximal joint member 62 is pivotally coupled to the distal joint member 64 about a pivot point “P” to define the joint 28. Since the joint members 62, 64 are fixedly coupled to respective outer shaft tubes 50 and 68, the outer shaft tubes 50 and 68 and the proximal and distal portions 20, 22 of the elongated shaft 18 may pivot relative to one another. Likewise, since the end effector 16 is coupled to the distal portion 22 of the elongated shaft 18, the end effector 16 may pivot relative to the longitudinal axis A-A.

Referring now to FIGS. 2C and 2D, the rotating collar 40 may be turned in the direction of arrow “R1” to pivot the outer shaft tube 68 in the direction of arrow “R2.” Rotating the collar 40 rotates the cam drive tube 44 and the cam member 52. As the cam member 52 rotates, the cam surface 54 urge the cam follower 58 in a distal direction The cam follower 58, in turn, engages the distal joint member 64 at a lateral distance from the pivot point “P” and urges the distal joint member 64 and outer shaft tube 68 in the direction of arrow “R2.” The outer shaft tube 68 pivots through an angle α relative to the longitudinal axis A-A. Although the angle α may vary, in one embodiment, an angle α of about 35° may be appropriate. For other embodiments, a negative angle α is also contemplated such that the outer shaft tube 68 may articulate in two directions. For example, the outer shaft tube may articulate from a negative angle (−α) to the left to a positive angle α to the right.

As the outer tube shaft 68 and the distal joint member 64 pivot in the direction of arrow “R2,” the distal joint member 64 urges the cam follower 56 in a proximal direction. The cam follower 56 maintains engagement with the sloped cam surface 54 of the cam member 52 as the cam follower 56 moves proximally. This continuous engagement eliminates slack or play in the articulation mechanism, and permits the rotation direction of the cam member 52 to be reversed at any time to induce an opposite longitudinal motion in cam followers 56, 58. Rotating the collar 40 in the direction of arrow “R3” drives rotation of the cam drive tube 44 and the cam member 52 in the direction of arrow “R3.” As the cam member 52 rotates, the cam surfaces 54 urge the cam follower 56 in a distal direction against the distal joint member 64 at a lateral distance from the pivot point “P.” The distal joint member 64 and outer shaft tube 68 are thus urged toward the longitudinal axis A-A to return to the orientation of FIG. 2A.

Referring now to FIGS. 3A through 30, various components of the elongated shaft 18 are configured to facilitate the articulation described above with reference to FIGS. 2A through 2D. The outer shaft tube 50 includes a lateral slot 70 extending therethrough, and the cam drive tube 44 includes a circular bore 72 therethrough. The circular bore 72 receives the pin 46 (FIG. 2B) in a friction fit or other suitable manner such that the rotational motion in the collar 40 may be transferred to the cam drive tube 44. The pin 46 extends through the slot 70, which provides clearance for the pin 46 to rotate through a fixed distance. Thus, the slot 70 defines the limits of travel for the collar 44 and provides positive stops for the elongated shaft 18 at the aligned configuration of FIG. 2A and the articulated configuration of FIG. 2C. Other embodiments may associate the slot 70 with an indexing mechanism (not shown) such that the pin 46 and, thus, the outer shaft tube 68 may have a tendency to stop at intermediate locations.

The proximal and distal joint members 62, 64 each include a body portion 72, 74 respectively, having a diameter sufficiently small to be received within the outer shaft tubes 50 and 68. The body portions 72, 74 may be friction fit within the outer shaft tubes 50, 68, fixedly attached with an appropriate adhesive or coupled to the outer shaft tubes 50, 68 in other suitable manners. Pivot flanges 76 and 78 extend from the body portions 72, 74 and are configured to abut an end of the respective outer shaft tube 50, 68 against a flat surface 76a, 78a. Thus, the pivot flanges 76, 78 protrude from the outer shaft tubes 50, 68. The pivot flange 76 of the proximal joint member 62 includes a bore 80 extending therethrough to receive a pair of bosses 82 projecting from the pivot flange 78 of the distal joint member 64 to pivotally couple the joint members 62, 64. The proximal joint member 62 includes the pair of guide slots 60 to receive the cam followers 56, 58 as discussed above. Each of the joint members 62, 64 also includes a central passageway, 84, 86 respectively, to permit passage of electrical cables, tensile control wires, cutting elements, or other components to facilitate operation of the end effector 16 (FIG. 1). The proximal and distal joint members 62, 64 may be constructed as molded plastic components.

The cam member 52 includes a body portion 88 having a diameter sufficiently small to be received within the cam drive tube 44 such the cam member 52 may be fixedly coupled to the cam drive tube 44. The cam member 52 also includes a central passageway 90 to permit passage of components for the operation of the end effector 16 (FIG. 1). The sloped forward cam surfaces 54 are shaped to provide sufficient travel to the cam followers 56, 58 to produce an appropriate angle α, as discussed above with reference to FIG. 2C, as the collar 44 is rotated through the range of motion defined by the slot 70 in the outer shaft tube 50.

The cam followers 56, 58 may constructed as be identical components. Each cam follower 56, 58 includes a curved face 92 at a proximal end to interface with the forward cam surfaces 54 of the cam member and a curved face 94 at a distal end to interface with flat cam surfaces 96 on the distal joint member 64. The curved faces 94 press against the flat cam surfaces 96 to induce pivotal motion in the outer shaft tube 68.

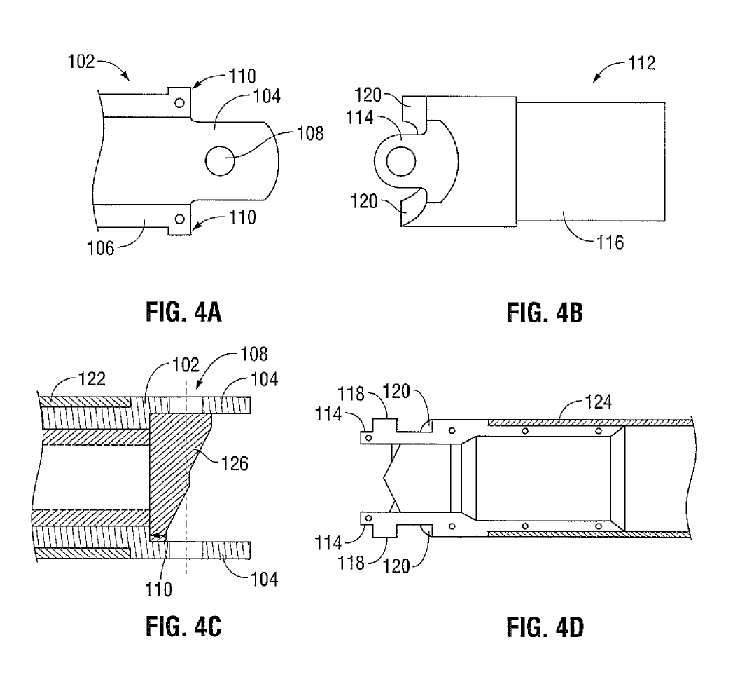

Referring now to FIGS. 4A through 4F, alternate embodiments of various components may also be assembled to form an articulating shaft. A proximal joint member 102 includes a pair of opposed pivot flanges 104 extending longitudinally from a body portion 106. The pivot flanges 104 are disposed at a laterally central location on the proximal joint member 102 and include bore 108 extending therethrough to define a pivot point. A flat bearing surface 110 is provided on each lateral side of the pivot flanges 104.

A distal joint member 112 includes a pair of opposed pivot flanges 114 extending longitudinally from a body portion 116. The pivot flanges 114 are disposed at a laterally central location on the distal joint member 112 and include pivot bosses 118 thereon for reception in the bore 108 of the proximal joint member 102. Sloped cam surfaces 120 are provided on each lateral side of the pivot flanges 114.

As depicted in FIGS. 4C and 4D, the proximal joint member 102 may be received in an outer shaft tube 122 and the distal joint member 112 may be received in an outer shaft tube 124. A cam member 126 may be received within the proximal joint member 102 to abut the flat bearing surface 110. The cam member 126 may be coupled to a cam drive tube and a rotating collar (not shown) in a similar manner to the elongated shaft 18 discussed with reference to FIG. 2A. Alternatively, the cam member 126 may extend directly to a rotating collar. Thus, the cam member 126 may be induced to rotate against the bearing surface 110 of the proximal joint member 102. When the bosses 118 of the distal joint member 112 are received within the bore 108 of the proximal joint member 102, the cam member 126 engages the sloped cam surfaces 120 of the distal joint member 112 as the cam member 126 rotates. This engagement causes the outer shaft tube 124 to pivot relative to the outer shaft tube 122.

As depicted in FIGS. 4E and 4F, the cam member 126 includes a pair of lateral wings 130 and 132 projecting from a body portion 134. The lateral wings 130, 132 include an underside 136, which engages the bearing surface 110 of the proximal joint member 102, and sloped cam surfaces 140, 142 which directly engage the sloped cam surfaces 120 of the distal joint member 112. The lateral wings 130, 132 project laterally such that the wings 130, 132 do not interfere with the pivot flanges 104, 114 of the proximal and distal joint members 102, 112 as the cam member 126 rotates.

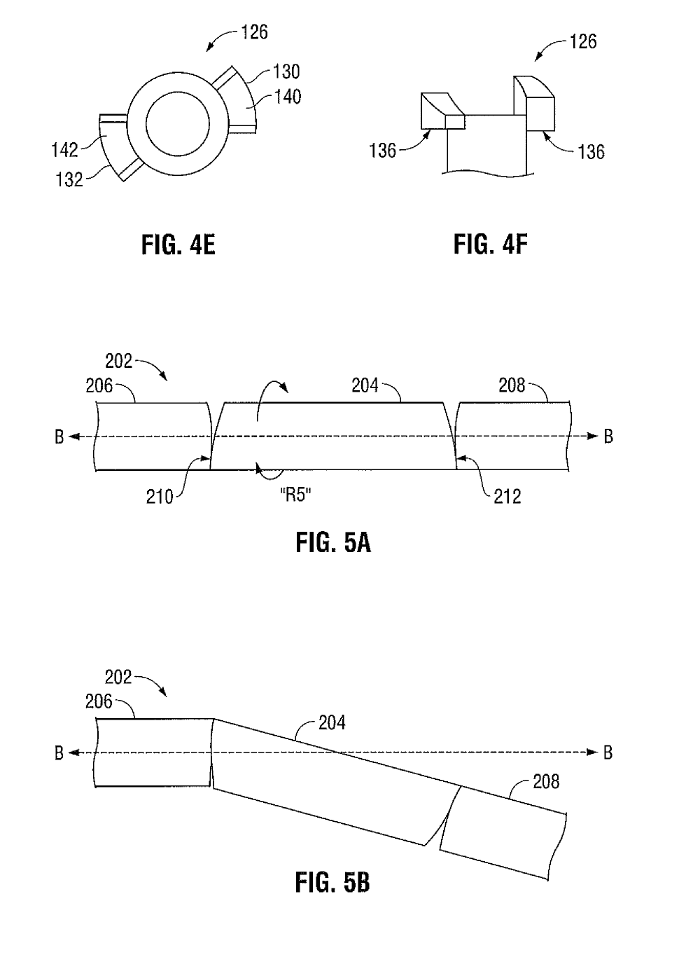

Referring now to FIGS. 5A and 5B, another embodiment of an elongated shaft is depicted generally as 202. The elongated shaft 202 defines a double-joint including a central segment 204 coupled between proximal and distal segments 206 and 208. The proximal segment 206 defines a longitudinal axis B-B. The central segment 204 engages the proximal and distal segments 206 and 208 across sloped edges 210, 212 such that the central segment 204 may act as a cam member to drive articulation. The central segment 204 may be coupled to a cam drive tube and a rotating collar (not shown) in a similar manner as in the elongated shaft 18 discussed with reference to FIG. 2A. Thus, the central segment 204 may be induced to rotate in the direction of arrows “R5.” Such a rotation will induce the articulation in the central and distal segments 202, 208 relative to the longitudinal axis “B” as depicted in FIG. 5B.

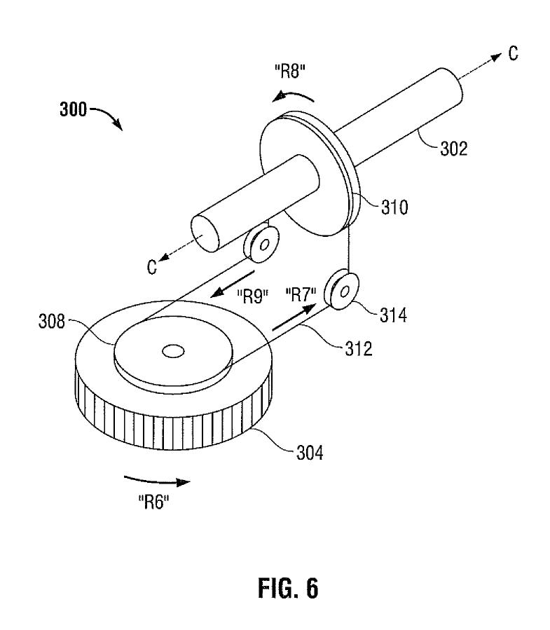

Referring now to FIG. 6, a pulley drive mechanism 300 may be employed to impart rotational motion to a cam drive tube 302. The cam drive tube 302 defines a longitudinal axis C-C, and may be coupled to a cam member such as cam member 52 as discussed above with reference to FIG. 2A. Rotational motion transmitted through the cam drive tube 302 may thus be employed to drive articulation of an elongated shaft such as the elongated shaft 18 as described above with reference to FIG. 2A.

The pulley drive mechanism includes a rotating collar 304, which may be mounted at an oblique orientation with respect to the longitudinal axis C-C. For some applications, this may permit the rotating collar 304 to be mounted in a more convenient or accessible location than mounting the collar 304 coaxially with the longitudinal axis C-C. The rotating collar is fixedly coupled to a drive pulley 308 such that rotational notion of the collar 304 is imparted to the drive pulley 308. Similarly, a follower pulley 310 is fixedly coupled to the cam drive tube 302 such that rotational motion in the follower pulley 310 is imparted to the cam drive tube 302. The drive pulley 308 is coupled to the follower pulley 310 with a cable, band or belt 312 such that the follower pulley 310 may be driven by the drive pulley 308. A pair of idler pulleys 314 engages the belt 312 to define an angle in the belt 310. The angle permits the collar 304 to be mounted obliquely with respect to the longitudinal axis C-C.

In use, a surgeon may rotate the rotating collar 304 in the direction of arrow “R6.” The drive pulley 308 also rotates in the direction of arrow “R6” and drives the belt 312 in the direction of arrow “R7.” The belt 312, in turn, drives follower pulley 310 in the direction of arrow “R8.” The cam drive tube 302 is also driven in the direction of arrow R8. Since the belt 310 returns to the drive pulley 308 in the direction of arrow “R9,” the collar 304 may be rotated in an opposite direction to drive the cam drive tube 302 in an opposite direction.

Although the foregoing disclosure has been described in some detail by way of illustration and example, for purposes of clarity or understanding, it will be obvious that certain changes and modifications may be practiced within the scope of the appended claims.

What is claimed is:

1. A surgical instrument comprising:
a handle adapted for manipulation by a user;
an elongated shaft extending distally from the handle, the elongated shaft including a proximal portion coupled to the handle and a distal portion pivotally coupled to the proximal portion, the proximal portion having a longitudinal axis defined therethrough;
an end effector coupled to the distal portion of the elongated shaft, the end effector articulatable relative to the longitudinal axis as the distal portion of the elongated shaft pivots relative to the proximal portion of the elongated shaft;
a cam member rotatably mounted about the longitudinal axis and configured to impart a force to the distal portion of the elongated shaft upon rotation of the cam member to effect pivotal motion of the distal portion of the elongated shaft; and
a rotating collar supported on the handle, the rotating collar operable to impart rotational motion to the cam member.

2. The instrument according to claim 1, wherein the rotating collar includes a rotating collar is coupled to a cam driver extending through the proximal portion of the elongated shaft, the cam driver configured to rotate about the longitudinal axis to impart rotational motion to the cam member.

3. The instrument according to claim 2, wherein the rotating collar is mounted for rotation about the longitudinal axis.

4. The instrument according to claim 2, wherein the rotating collar is mounted with an oblique orientation with respect to the longitudinal axis.

5. The instrument according to claim 4, wherein the rotating collar is coupled to a drive pulley, the cam driver is coupled to a follower pulley, and the follower pulley is coupled to the drive pulley by a flexible belt.

6. The instrument according to claim 1, wherein the proximal portion of the elongated shaft includes a proximal joint member at a distal end thereof, and the distal portion of the elongated shaft includes a distal joint member at a proximal end thereof, the distal joint member pivotally coupled to the proximal joint member, and wherein the proximal and distal joint members exhibit a molded plastic construction.

7. The instrument according to claim 1, wherein the cam member is operable to engage a first cam follower such that the first cam follower is adapted to move longitudinally in a distal direction in response to rotational motion of the cam member about the longitudinal axis in a first direction, and wherein the first cam follower is adapted to engage the distal portion of the elongated shaft at a lateral distance from a pivot point to impart the force to distal portion of the elongated shaft.

8. The instrument according to claim 7, wherein the cam member is adapted to engage a second cam follower such that the second cam follower is adapted to move longitudinally in a distal direction in response to rotational motion of the cam member about the longitudinal axis in a second direction, and wherein the second cam follower is adapted to engage the distal portion of the elongated shaft at a lateral distance from the pivot point on an opposite side of the pivot point in relation to the first cam follower to impart the force to the distal portion of the elongated shaft.

9. The instrument according to claim 8, wherein the first and second cam followers are adapted to maintain engagement with both the cam member and the distal portion of the elongated shaft as the cam member rotates in the first and second directions.

10. The instrument according to claim 2, wherein the cam driver is operable to rotate within an outer shaft tube having a lateral slot defined therein, and wherein the cam driver is coupled to a pin projecting through the lateral slot such that the lateral slot defines the limits of travel for the collar.

11. The instrument according to claim 1, wherein the elongated shaft defines a double-joint wherein the cam member defines a central segment coupled between proximal and distal segments such that the proximal and distal segments pivot relative to the central segment upon rotation of the central segment.

12. The surgical instrument according to claim 1, wherein the end effector includes a pair of jaw members, and wherein at least one jaw member is configured to move between an open position substantially spaced from the other of the pair of jaw members and a closed position wherein the jaw members are closer together.

13. The surgical instrument according to claim 12, wherein at least one of the pair of jaw members is coupled to a source of electrical energy.

14. An electrosurgical instrument, comprising:
a handle adapted for manipulation by a user;
an elongated shaft extending distally from the handle and having a longitudinal axis defined therethrough;
an end effector pivotally coupled to a distal end of the elongated shaft such that the end effector may articulate relative to the longitudinal axis, the end effector including a pair of jaw members wherein at least one jaw member is configured to move between an open position substantially spaced from the other of the pair of jaw members and a closed position wherein the jaw members are closer together, and wherein at least one of the jaw members is connectable to a source of electrosurgical energy;
a cam member rotatably mounted about the longitudinal axis adjacent the distal end of the elongated shaft, the cam member configured to articulate the end effector upon rotation of the cam member;
a cam driver rotatably mounted about the longitudinal axis and extending between the handle and the cam member, the cam driver coupled to the cam member to impart rotational motion thereto; and
a rotating collar supported on the handle, the rotating collar operable to impart rotational motion to the cam driver.
